(19) United States
(12) Patent Application Publication
(10) Pub. No.: US 2022/0204917 A1
(43) Pub. Date: Jun. 30, 2022

(54) PRODUCTION OF FUNCTIONAL PROTEIN USING MICROALGAE IN MIXOTROPHIC AND/OR HETEROTROPHIC CULTIVATION

(71) Applicant: Sophie?s BioNutrients Pte. Ltd., Singapore (SG)

(72) Inventor: Yao-Hsin Wang, Singapore (SG)

(73) Assignee: Sophie?s BioNutrients Pte. Ltd., Singapore (SG)

(21) Appl. No.: 17/563,067

(22) Filed: Dec. 28, 2021

Related U.S. Application Data
(60) Provisional application No. 63/132,822, filed on Dec. 31, 2020.

(51) Int. Cl. C12N 1/12; C12M 1/00

(52) U.S. Cl. CPC C12N 1/12; C12R 2001/89; C12M 31/10; C12M 21/02

(57) ABSTRACT

A method executed by a bioreactor system to cultivate a microalgae comprising a photoreceptor sensitive to a region of a visible spectrum under an autotrophic condition and a fermentation condition is described. A first process incubates the microalgae under the autotrophic condition and the fermentation condition simultaneously to allow the microalgae to grow and then removes the fermentation condition halfway through the process. A second process incubates the microalgae under the autotrophic condition and the fermentation condition simultaneously to allow the microalgae to grow. Both processes generate compounds, or functional proteins.

### CROSS-REFERENCE TO RELATED APPLICATIONS SECTION

This present application is a US Non-Provisional application, which claims benefit from U.S. Provisional Application No. 63/132,822 filed on Dec. 31, 2020, the entire contents of which are hereby incorporated by reference in their entirety.

### FIELD OF THE EMBODIMENTS

The field of the invention and its embodiments relate to methods and systems for the production of functional proteins using microalgae in mixotrophic and/or heterotrophic cultivation.

### BACKGROUND OF THE EMBODIMENTS

Algae are photosynthetic organisms that grow in a range of aquatic habitats, including lakes, pounds, rivers, and oceans. Algae can tolerate a wide range of temperatures, salinities, and pH values, as well as differing light intensities. Additionally, algae may also grow alone or in symbiosis with other organisms. Algae may be broadly classified as Rhodophyta (red algae), Phaeophyta (brown algae), or Chlorophyta (green algae). Algae may be further classified by size, as macroalgae (which are multicellular, large-size algae that are visible with the naked eye) or microalgae (which are microscopic, single cells that may be prokaryotic or eukaryotic).

Currently, there are an estimated 300,000 to 1 million species of microalgae in existence. Microalgae has recently attracted considerable interest due to their extensive applications in the renewable energy field, the biopharmaceutical field, and the nutraceutical field. Specifically, microalgae may be a sustainable and economical source of biofuels, bioactive medicinal products, and food ingredients. Moreover, microalgae also have applications in wastewater treatment and atmospheric CO₂ mitigation. Thus, microalgae produces a wide range of bioproducts, including polysaccharides, lipids, pigments, proteins, vitamins, bioactive compounds, and antioxidants.

Chlorella is a genus of single-celled green algae belonging to the division Chlorophyta. Chlorella I spherical in shape, about 2 to 10 μm in diameter, and is without flagella. It contains the green photosynthetic pigments chlorophyll-a and -b in its chloroplast. Chlorella multiples rapidly, requiring only carbon dioxide, water, sunlight, and a small amount of minerals to reproduce.

Chlorella is a potential food source since it is high in protein and other essential nutrients. For example, when dried, Chlorella contains about 45% protein, 20% fat, 20% carbohydrate, 5% fiber, and 10% minerals and vitamins (e.g., vitamin B12, vitamin C, iron, magnesium, zinc, copper, potassium, and/or calcium, etc.). Due to this, Chlorella has been labeled as a “superfood” and has garnished significant attention from the vegan community. Further, Chlorella has been explored as a potential source of food and energy because its photosynthetic efficiency can, in theory, reach 8%, which exceeds that of other highly efficient crops, such as sugar cane.

Although microalgae are feasible sources for bioenergy and biopharmaceuticals in general, some limitations remain. One such limitation to microalgae cultivation is light intensity. Light duration and intensity directly affect the photosynthesis of microalgae. At very low and very high light intensities, microalgae cannot grow efficiently. Higher light intensities will increase a photosynthetic rate of the microalgae to a maximum point, after which it levels off until the photosynthetic rate is balanced by photorespiration and photoinhibition. Photorespiration refers to a process in plant metabolism, where the enzyme, ribulose-1,5-bisphosphate carboxylase/oxygenase (RuBisCO), oxygenates ribulose 1,5-bisphosphate (RuBP), wasting some of the energy produced by photosynthesis. Photoinhibition is light-induced reduction in the photosynthetic capacity of a plant, alga, or cyanobacterium. Essentially, optimal light intensity needs to be determined experimentally in each case to maximize CO₂ assimilation and minimize both photorespiration and photoinhibition. Uniform distribution of light is also needed to avoid photoinhibition.

Another limitation to the growth of microalgae is temperature. Each species of microalgae has its own optimal growth temperature. Increasing a temperature to the optimum range exponentially increases algal growth, but an increase or decrease in the temperature beyond the optimal point retards or even stops algae growth and activity. The optimum temperature range for most algal species is 20-30° C. Growing microalgae cultures at non-optimal temperatures will result in high biomass losses.

A further limitation to microalgae growth involves ensuring the nutritional needs of the microalgae are met. Typically, all strains of microalgae have the following backbone: nitrogen, phosphorus, and carbon (CH_{1.7} O_{0.4} N_{0.15} P_{0.0094}). Some marine microalgae species also require silicon as a macronutrient. Specifically, quantities of the available nitrogen in the culture directly alter cell growth. Nitrogen limitation in the microalgae culture can reduce growth and biomass productivity, however, can increase production of carbohydrates and lipids. As an illustrative example, an optimum concentration of nitrogen for Chlorella vulgaris is 0.5 g/l, at which it produces 3.43 g/l biomass. Moreover, the micronutrients molybdenum (Mo), potassium (K), cobalt (Co), iron (Fe), magnesium (Mg), manganese (Mn), boron (B), and zinc (Zn) are only required in trace amounts, but have been shown to have a strong impact on microalgae growth, as they influence many enzymatic activities in algal cells. Nutrient deficiency greatly affects the microalgae growth rate and results in low biomass.

With increasing attention being paid to the consumption of healthy nutritional foods, algal protein has moved to the forefront of non-animal protein sources. However, the applications of Chlorella protein as a functional ingredient in food still requires further exploration. Thus, a need exists for methods and systems for the production of functional proteins using microalgae in mixotrophic and/or heterotrophic cultivation.

### Review of Related Technology:

EP 3,074,522 B1 describes a method for carotenoid enrichment and protein enrichment of a heterotrophically cultivated biomass of a microalgae of the Chlorella genus. The method includes: culturing the microalgae in a minimum medium supplemented with a nitrogen source in an organic form. The nitrogen source is added in an amount such that nitrogen in the organic form does not exceed 10% of the total nitrogen contained in the fermentation medium. This reference also appreciates that the exploitation of microalgae generally requires the control of the fermentation conditions allowing to accumulate its components of interest, such as lutein.

CN 107075547 A describes methods the protein-enriched of Chlorella microalgae cultivated under the conditions of heterotrophism, where the fermentation medium lacks a nitrogen-free nutrient source.

JP 2016/514471 A describes a protein enrichment method for microalgae cultured under heterotrophic conditions. The microalgae is of the genus Chlorella. The heterotrophic culture is caused by the lack of nitrogen-free nutrient sources in the fermentation medium. This reference also explains that the yield of lutein by heterotrophically growing Chlorella protothecoides increases when the production temperature increases from 24° C. to 35° C.

WO 2015079182 A1 relates to a process for the enrichment, with carotenoids and proteins, of a biomass of a microalgae cultivated under heterotrophic conditions. The microalgae is of the Chlorella genus. The method includes: culturing the microalgae in a minimum medium supplemented with a nitrogen source in an organic form, such as: yeast extract and/or corn steep liquor.

U.S. Pat. No. 3,108,402 A relates to methods of producing carotenoids, and more specifically, to improved methods for the production of beta-carotene and xanthophyll by the cultivation of a grass green algae of the botanical division Ohlorophyta. This reference also describes methods involving the cultivation of algae under heterotrophic conditions in an aqueous nutrient medium.

U.S. Pat. No. 2,949,700 A relates to methods for the production of carotenoids. More specifically, this invention relates to an improved method for the production of beta-carotene and xanthophyll by the cultivation of a grass green algae of the botanical division Chlorophyta. This reference also describes methods involving cultivation of algae in an aqueous organic medium using urea as a supplementary source of nitrogen.

U.S. Pat. No. 3,142,135 A relates to methods for the production of carotenoids. More specifically, this invention relates to an improved method for the production of betacarotene and xanthophyll by the cultivation of a grass green algae of the botanical division Chlorophyta. This reference also relates to a method involving cultivation of algae in an aqueous organic medium under conditions resulting in maximum carotenoid production.

FR 3,003,873 A1 relates to a protein enrichment method of a microalgae grown in heterotrophy. The heterotrophic culture comprises a step for limiting the growth of the microalgae by a deficiency of the medium of fermentation in a non-nitrogenous nutritional source.

U.S. Published Patent Application No. 2019/0254291 A1 and U.S. Published Patent Application No. 2019/0254292 A1 describe compositions of microalgae-derived flour from multiple genera, species, and strains of edible microalgae. Microalgae used in these references are free of algal toxins and contain varying levels of primarily monounsaturated triglyceride oil. Flours disclosed in these references are formulated as free flowing blendable powders, mixed food ingredients, oxidation stabilized, homogenized and micronized, and combinations therein. Flours disclosed in these references are also form self-stabilizing emulsions in slurries with manageable viscosities. These references also describe methods to formulate flours and methods for incorporating them into food compositions. In some cases, the biomass has between 20-115 μg/g of total carotenoids, including 20-70 μg/g lutein.

JP 2014/061009 A describes algal biomass, algal oil, food compositions comprising microalgal biomass, whole microalgal cells, and/or microalgal oil in combination with one or more other edible ingredients, and methods of making such compositions by combining algal biomass or algal oil with other edible ingredients. In preferred embodiments, the microalgal components are derived from microalgal cultures grown and propagated heterotrophically, in which the algal cells comprise at least 10% algal oil by dry weight. In some examples, the total carotenoid amount comprises 0 to 70 mcg/g lutein.

KR 101914694 B1 describes a food composition comprising algae biomass or algae powder having a high lipid content. In some instances, the biomass or algae powder has a total carotenoid amount from about Om to about 115 μg, including 20-70 μg lutein per gram of microalgae biomass or algae powder.

Various methods are known in the art. However, their means of operation are substantially different from the present disclosure, as the other inventions fail to solve all the problems taught by the present disclosure.

### SUMMARY OF THE EMBODIMENTS

The present invention and its embodiments provide methods and systems for the production of functional proteins using microalgae in mixotrophic and/or heterotrophic cultivation.

A first embodiment of the present invention describes a method executed by a bioreactor system to cultivate a microalgae comprising a photoreceptor sensitive to a region of a visible spectrum. The bioreactor system includes a bioreactor having one or more light sources implanted into one or more holes of the bioreactor, a culture media located inside of the bioreactor that comprises a carbon source, and an irradiance of light from the one or more light sources that includes the region of the visible spectrum in a sufficient intensity to transduce the photoreceptor.

The method includes incubating the microalgae under the autotrophic condition and the fermentation condition simultaneously to allow the microalgae to grow and removing the fermentation condition halfway through the process. Next, the method includes generating compounds (e.g., functional proteins) during growth of the microalgae.

In some examples, the method further includes producing a material from the microalgae, where the material includes a polysaccharide, a pigment, a lipid, and/or a hydrocarbon. The method may also further include recovering the material, extracting the material, and/or processing the material to form another material. The other material may be a fuel, biodiesel, jet fuel, a cosmetic, a pharmaceutical agent, a surfactant, and/or a renewable diesel.

A second embodiment of the present invention describes a method executed by a bioreactor system to cultivate a microalgae comprising a photoreceptor sensitive to a region of a visible spectrum. The bioreactor system includes a bioreactor having one or more light sources implanted into one or more holes of the bioreactor, a culture media located inside of the bioreactor that comprises a carbon source, and an irradiance of light from the one or more light sources that includes the region of the visible spectrum in a sufficient intensity to transduce the photoreceptor. The method includes incubating the microalgae under the autotrophic condition and the fermentation condition simultaneously to allow the microalgae to grow and generating compounds (e.g., functional proteins) during growth of the microalgae.

### BRIEF DESCRIPTION OF THE DRAWINGS

FIG. 1 depicts a schematic block diagram of two methods to produce functional proteins using microalgae in mixotrophic and/or heterotrophic cultivation, according to at least some embodiments described herein.

### DESCRIPTION OF THE PREFERRED EMBODIMENTS

The preferred embodiments of the present invention will now be described with reference to the drawings. Identical elements in the various FIGURES are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

Microalgae may comprise a photoreceptor sensitive to a region of a visible spectrum. As defined herein, a “microalgae” refers to a eukaryotic microbial organism that contains a chloroplast, and optionally, that is capable of performing photosynthesis, or a prokaryotic microbial organism capable of performing photosynthesis. A culture media may comprise a carbon source and may be located inside of a bioreactor or a photobioreactor. As described herein, a “bioreactor” is an enclosure or partial enclosure, in which cells are cultured, and optionally in suspension. As described herein, a “photobioreactor” refers to a container, at least part of which is at least partially transparent or partially open, thereby allowing light to pass through, in which one or more microalgae cells are cultured.

The microalgae may be located in the culture media. The microalgae may be subjected to numerous conditions, such as light, stress conditions, and carbon dioxide (CO₂). The microalgae is configured to convert the atmospheric CO₂ to raw materials (e.g., the biomass) via use of the light. The biomass may include: proteins, carbohydrates, and lipids and may be directly used for food supplements and/or human food, among other uses not explicitly listed herein.

The specific process by which the microalgae converts the atmospheric CO₂ to the raw materials (e.g., the biomass) may include a process known as oxygenic photosynthesis. During this process, water is the electron donor, and oxygen is released after hydrolysis. The equation for photosynthesis can be written as follows:

H₂O+CO₂+Photons(light)→[CH₂O]ₙ+O₂ [Equation 1]

The reaction of Equation 1 can be divided into two pathways: (1) a light-dependent reaction and (2) a dark or light-independent reaction. The light-dependent reaction involves both photochemical and redox reaction steps. The overall equation for the light-dependent reaction includes:

2H₂O+2NADP⁺+3ADP+3P+light→2NADPH+2H⁺+3ATP+O₂ [Equation 2]

In this reaction, ADP refers to adenosine diphosphate, P refers to the element phosphate, and NADP refers to nicotinamide adenine dinucleotide phosphate. Light energy is used to synthesize ATP and the NADPH (e.g., the reduced form of NADP⁺), which are energy storage molecules.

In the light-independent reaction, RuBisCO captures CO₂ from the atmosphere. This process requires the newly formed NADPH, called the Calvin cycle or the Benson-Calvin cycle. As defined herein, the “Benson-Calvin cycle” is a set of chemical reactions that take place in chloroplasts during photosynthesis. To be more specific, carbon fixation produces an intermediate product that is then converted into the final carbohydrate products. The carbon skeletons that are produced by photosynthesis are then used in a variety of processes to form other organic compounds. The overall equation for the light-independent reaction includes:

3CO₂+9ATP+6NADPH+6H⁺→C₃H₆O₃-phosphate+9ADP+8P+6NADP⁺+3H₂O [Equation 3]

The fixation or reduction of CO₂ takes place by combining CO₂ with a five-carbon sugar, ribulose 1,5-bisphosphate (Ru5BP), generating two molecules of a three-carbon compound, glycerate 3-phosphate (GP). In the presence of ATP and NADPH (from the light-dependent stages), GP is reduced to glyceraldehyde 3-phosphate (G3P) (also called 3-phosphoglyceraldehyde (PGAL) or triose phosphate). Most of the G3P that is produced is used to regenerate Ru5BP so that the process can continue. Of the six molecules of G3P, one is not “recycled” and often condenses to form hexose phosphate, yielding sucrose, starch, and cellulose. The sugars that are produced during carbon metabolism yield carbon skeletons that can be used for other metabolic reactions, such as the production of amino acids and lipids.

The biomass may be converted to biofuels and/or bio-products. Examples of the biofuels include solid biofuels, liquid biofuels, and/or gas biofuels. An example of the solid biofuels may include bio-char. Examples of the liquid biofuels may include bioethanol, biodiesel, vegetable oil, etc. Examples of the gas biofuels may include biohydrogen and/or biosyngas. It should be appreciated that the examples of the biofuels are not limited to those examples explicitly listed herein. Non-exhaustive examples of the bio-products may include: poly-unsaturated fatty acids, antioxidants, coloring agents, vitamins, anti-cancer drugs, anti-microbial drugs, etc.

It should be appreciated that in some examples of the instant disclosure, the process of fermentation of the microalgae may be used to convert algal sugars (such as glucose, fructose, maltose, and/or rhamnose, among other examples not explicitly listed herein) to the liquid biofuels of bioethanol. As defined herein, “fermentation” refers a metabolic process that produces chemical changes in organic substrates through the action of enzymes. In the context of food production, “fermentation” may refer to any process in which the activity of microorganisms brings about a desirable change to a foodstuff or beverage. In microorganisms, fermentation is the primary means of producing adenosine triphosphate (ATP) by the degradation of organic nutrients anaerobically. As an example, fermentation may be used to produce alcoholic beverages, such as wine and beer.

The yield and quality of the bioethanol (e.g., the liquid biofuels) produced is strongly dependent on the fermentation process, which is affected by several factors, such as temperature, pH, oxygen, substrate concentration, and the fermenter organism used. In some examples, the microalgae may be used for food applications. In other examples, the microalgae may be used for pharmaceuticals, antibiotics, plastics replacements, cosmetics, nutritional supplements, flavoring, color pigments, biofuel, cooking oil, etc.

The bioreactor for cultivating microalgae may be the photobioreactor. A specific example of the bioreactor is a closed bioreactor, such as a fermentation vessel or tank. In some examples, the fermentation vessel or tank may be a cylindrical-conical fermentation tank. However, it should be appreciated that the bioreactor may be any type of bioreactor. The bioreactor may include a glass, a metal, or a plastic tank equipped with gauges and settings to control aeration, stir rate, temperature, pH, and other parameters not explicitly listed herein. Generally the gauges and settings are operatively coupled to the bioreactor. In some examples, the bioreactor may be small in size (e.g., 5-10 L or less) to accommodate bench-top applications. In other examples, the bioreactor may be larger in size (e.g., 120,000 L or larger) for use in large-scale industrial applications.

The bioreactor may have one or more ports to allow entry of gases, solids, semi-solids and/or liquids into the chamber containing the microalgae. The ports may be attached to tubing or other means of conveying substances. Gas ports, for example, convey gases into the culture media. In some examples, a gas content of the bioreactor may be modified based on the growth of the specific microorganism. In other examples, part of the volume of the bioreactor may contain gas, rather than a liquid. The bioreactor may include gas inlets used to pump gases into the bioreactor. Such gases may include air, air/O₂ mixtures, and noble gases, among other gases not explicitly listed herein.

Pumping gases into a bioreactor can feed cells O₂ and other gases, and may also aerate the culture, and therefore, generate turbidity. Increasing gas flow into the bioreactor may increase the turbidity of a culture of the microalgae. Placement of ports conveying gases into the bioreactor can also affect the turbidity of a culture at a given gas flow rate. Air/O₂ mixtures can be modulated to generate optimal amounts of O₂ for maximal growth by a particular organism. Microalgae grow significantly faster in the light under, for example, 3% O₂/97% air than in 100% air.

Moreover, the bioreactor may have one or more ports that allow media entry and/or for sampling the culture. In some examples, a sampling port can be used repeatedly without altering the axenic nature of the culture. The sampling port can be configured with a valve or other device that allows the flow of sample to be stopped and started. Alternatively, the sampling port can allow continuous sampling.

According to some examples, a bioreactor system for cultivating microalgae is described. The bioreactor system may comprise the bioreactor. The bioreactor may comprise one or more holes. A culture media may be located inside the bioreactor. The culture media may be a liquid medium. In examples, the culture media may contain components such as, a fixed nitrogen source, trace elements, a buffer for pH maintenance, and phosphate. The culture media may also include a fixed carbon source, such as: glucose, fructose, sucrose, galactose, xylose, mannose, rhamnose, N-acetylglucosamine, glycerol, floridoside, glucuronic acid, corn starch, depolymerized cellulosic material, sugar cane, sugar beet, lactose, milk whey, and/or molasses, among other examples not explicitly listed herein. In additional examples, the carbon source may be preferably glucose. The carbon source can be supplied at a concentration of less than 50 μM, at least about 50 μM, at least about 100 μM, at least about 500 μM, at least about 5 mM, at least about 50 mM, at least about 500 mM, and more than 500 mM of one or more exogenously provided fixed carbon source(s). Additional trace elements may be present in the culture media, such as: zinc, copper, cobalt, boron, manganese, and/or molybdenum.

The microalgae comprising a photoreceptor sensitive to a region of a visible spectrum may be located in the culture media. In some examples, the microalgae is of a mixotrophic strain. In examples, the microalgae may be adapted for both autotrophic growth and heterotrophic growth during a time period. According to some examples, the microalgae may be a Botryococcus sudeticus strain, a Botryococcus strain, a Neochloris oleabundans strain, a Neochloris strain, a Chlamydomonas reinhardtii strain, and/or a Chlamydomonas strain, among other examples not explicitly listed herein.

Exposure of the microalgae to one or more light sources, even in the presence of a fixed carbon source that the cells transport and utilize, can accelerate growth compared to culturing cells of the microalgae in the dark. As such, one or more light sources may be impregnated or implanted into each of the one or more holes of the bioreactor. Each of the one or more light sources may produce an irradiance of light in a full spectrum of light. In other examples, each of the one or more light sources may produce the irradiance of the light at a specific wavelength or in a region of the visible spectrum in a sufficient intensity to transduce the photoreceptor of the microalgae.

In some examples, each of the one or more light sources may include a natural light source (e.g., sunlight collected by a solar collector and transmitted to the interior of the bioreactor via an optical fiber) or an artificial light source. In other examples, the artificial light source may include a light-emitting diode (LED). In additional examples, the artificial light source may include a fluorescent tube. In some examples, the one or more light sources may include a combination of the natural light source and the artificial light source. Since uniform distribution of the one or more light sources is essential to avoid photoinhibition, the one or more holes of the bioreactor may be evenly distributed. It should be appreciated that the one or more light sources may be configured to supply a full spectrum or a specific wavelength of artificial light to a bioreactor, based on the specific application.

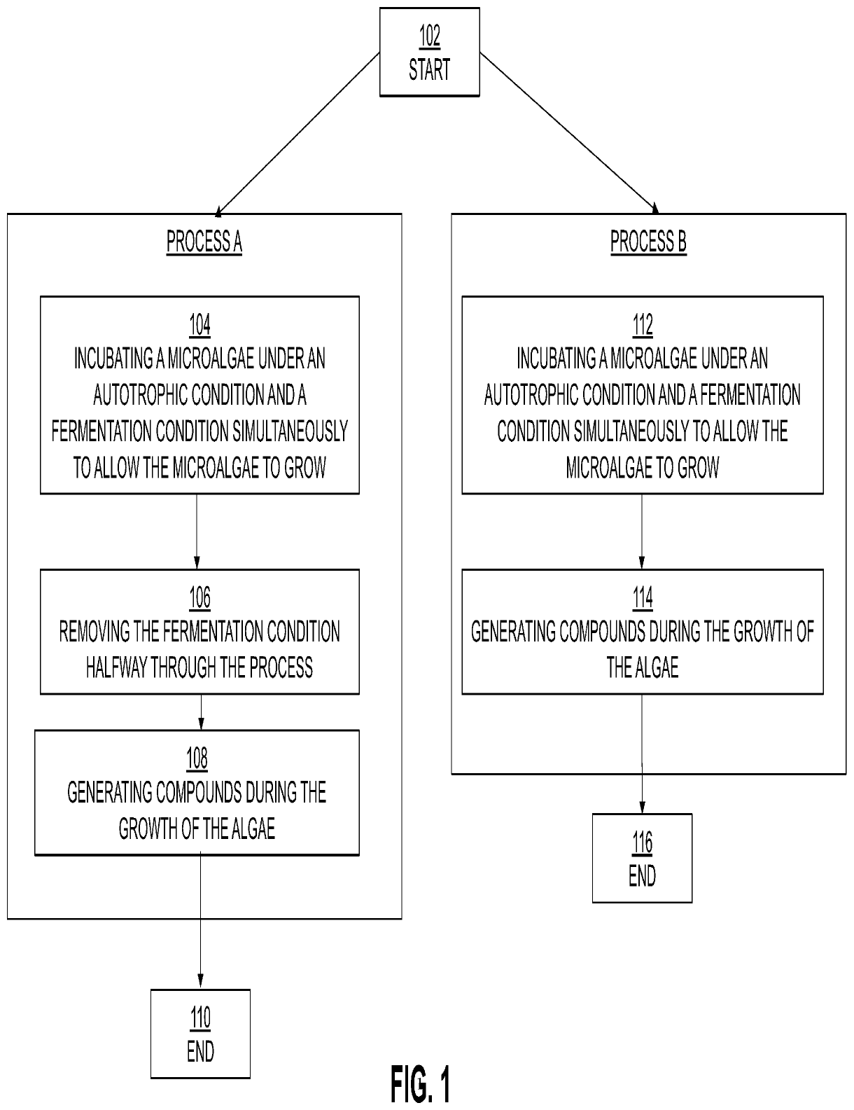

In some examples, two types of mixotrophic operations are performed for the fermentation of the microalgae. Such processes are depicted in FIG. 1. The method of FIG. 1 begins at a process step 102. For a first process (e.g., a process A), a process step 104 follows the process step 102 and includes incubating a microalgae under an autotrophic condition and a fermentation condition simultaneously to allow the microalgae to grow. Next, a process step 106 follows the process step 104 and includes removing the fermentation condition halfway through the process A. Next, a process step 108 follows the process step 106 and includes generating compounds (e.g., functional proteins) during the growth of the algae. A process step 110 ends the process A.

A second process (e.g., a process B) is also depicted in FIG. 1. A process step 112 follows the process step 120 and includes incubating a microalgae under the autotrophic condition and the fermentation condition simultaneously to allow the microalgae to grow. A process step 114 follows the process step 112 and includes generating the compounds (e.g., the functional proteins) during the growth of the algae. A process step 116 follows the process step 114 and ends the process B. It should be appreciated that the compounds (e.g., the functional proteins) produced by the process A differ from the compounds (e.g., the functional proteins) produced by the process B. For example, the process A may be used to increase a pace of growth of the microalgae.

As defined herein, an “autotroph” refers to an organism that is capable of synthesizing its own food from inorganic substances, using light or chemical energy. As defined herein, a “heterotroph” refers to an organism that cannot synthesize its own food and is dependent on complex organic substances for nutrition. As defined herein, a “mixotrophic strain” is defined as a strain of an organism that allows it to be both autotrophic and heterotrophic at the same time.

Proteins are macromolecules consisting of one or more long chains of amino acid residues. Proteins perform a vast array of functions within organisms, including catalyzing metabolic reactions, DNA replication, responding to stimuli, providing structure to cells, and organisms, and transporting molecules from one location to another. Proteins differ from one another primarily in their sequence of amino acids, which is dictated by the nucleotide sequence of their genes, and which usually results in protein folding into a specific three-dimensional structure that determines its activity.

Amino acids are the basic building blocks of the body and are organic compounds that contain amine (—NH2) and carboxyl (—COOH) functional groups, along with a side chain (R group) specific to each amino acid. In the form of proteins, amino acid residues form the second-largest component (water is the largest) of human muscles and other tissues. Amino acids are extremely versatile and more than 200 different amino acids exist. The most commonly known are the 22 proteinogenic amino acids.

Amino acids prove to be beneficial in numerous fields. For example, L-methionine and L-arginine work together with Glucosamine, Chondroitin, omega-3 and Methyl sulfonylmethane (MSM) to prevent and treat arthritis. L-glutamine, L-arginine and L-cysteine may be useful to improve one's immune system. Branched-chain amino acids (BCAAs) and especially L-leucine are essential for growth, recovery and maintenance of all muscle tissue. L-arginine, L-methionine, L-cysteine, L-lysine, L-glycine and L-proline boost ones natural skin and nail beauty.

L-arginine, L-carnitine and L-cysteine can significantly improve sperm quality and therefore male fertility. L-cysteine, L-glutathione and L-carnitine are powerful antioxidants, which protect ones cells from oxidative stress caused by free radicals. L-arginine and Pine bark Extract improve circulation throughout and protect ones body's arterial walls. Managing L-tryptophan levels can be good for ones sleep. BCAAs, L-glutamine and L-glycine reduce the risk of inflammatory diseases and chronic pain by strengthening ones immune system.

Magnesium, phytoestrogens and L-arginine help manage menopause by reducing hot flushes. L-arginine, L-lysine, zinc and vitamin C improve digestion and protect one from rectal diseases. L-arginine and Ginkgo biloba improve blood circulation, increasing oxygen and nutrient availability within the ear. Moreover, one may face a reduced risk of diabetes with L-arginine and L-carnitine, zinc, magnesium, chromium and omega-3.

In some examples, the microalgae may be used for the production of a high-value carotenoid, lutein, by heterotrophic fermentation. Lutein is a xanthophyll and one of 600 known naturally occurring carotenoids. Lutein is synthesized only by plants, and like other xanthophylls is found in high quantities in green leafy vegetables such as spinach, kale and yellow carrots. In green plants, xanthophylls act to modulate light energy and serve as non-photochemical quenching agents to deal with triplet chlorophyll (an excited form of chlorophyll), which is overproduced at very high light levels, during photosynthesis.

Effects of two oxidant-forming reactive oxygen species (ROS) on the biomass concentration, and yield and content of lutein in batch culture of heterotrophic Chlorella protothecoides were investigated in a study. See, Dong Wei, et al., “Enhanced production of lutein in heterotrophic Chlorella protothecoides by oxidative stress,” Science in China Series C: Life Sciences, 2008, Vol. 51, Pages 1088-1093, the entire contents of which are hereby incorporated by reference in their entirety. As defined herein, a “ROS” is a highly reactive chemical molecules formed due to the electron acceptability of O₂. The results of this study indicated that ¹O₂ could promote lutein formation and enhance lutein production in heterotrophic Chlorella protothecoides. Moreover, ¹O₂ produced from the reaction of H₂O₂ and NaClO was more effective in enhancing lutein production and reducing biomass loss than .OH from the reaction of H₂O₂ or NaClO plus Fe²⁺.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method executed by a bioreactor system to cultivate a microalgae comprising a photoreceptor sensitive to a region of a visible spectrum under an autotrophic condition and a fermentation condition, the bioreactor system comprising a bioreactor having one or more light sources implanted into one or more holes of the bioreactor, the method comprising:
incubating the microalgae under the autotrophic condition and the fermentation condition simultaneously to allow the microalgae to grow;
removing the fermentation condition halfway through the process; and
generating compounds during growth of the microalgae.

2. The method of claim 1, wherein the bioreactor system further comprises:
a culture media located inside of the bioreactor that comprises a carbon source, and
an irradiance of light from the one or more light sources that includes the region of the visible spectrum in a sufficient intensity to transduce the photoreceptor.

3. The method of claim 1, further comprising:
producing a material from the microalgae.

4. The method of claim 4, wherein the material is selected from the group consisting of: a polysaccharide, a pigment, a lipid, and a hydrocarbon.

5. The method of claim 3, further comprising:
recovering the material; and
extracting the material.

6. The method of claim 5, further comprising:
processing the material to form another material, wherein the other material is selected from the group consisting of: a fuel, biodiesel, jet fuel, a cosmetic, a pharmaceutical agent, a surfactant, and a renewable diesel.

7. The method of claim 1, wherein the compounds comprise functional proteins.

8. A method executed by a bioreactor system to cultivate a microalgae comprising a photoreceptor sensitive to a region of a visible spectrum under an autotrophic condition and a fermentation condition, the bioreactor system comprising a bioreactor having one or more light sources implanted into one or more holes of the bioreactor, the method comprising:
incubating the microalgae under the autotrophic condition and the fermentation condition simultaneously to allow the microalgae to grow; and
generating compounds during growth of the microalgae.

9. The method of claim 8, wherein the bioreactor system further comprises:
a culture media located inside of the bioreactor that comprises a carbon source, and
an irradiance of light from the one or more light sources that includes the region of the visible spectrum in a sufficient intensity to transduce the photoreceptor.

10. The method of claim 8, wherein the compounds comprise functional proteins.
